(19) United States
(12) Patent Application Publication
(10) Pub. No.: US 2008/0166472 A1
(43) Pub. Date: Jul. 10, 2008

(54) EVAPORATION PROCESS FOR SOLID PHASE MATERIALS

(75) Inventors: Peter B. MacKenzie, Kingsport, TN (US); Hitoshi Yamamoto, Pennington, NJ (US); Michaeal S. Weaver, Princeton, NJ (US); Vadim I. Adamovich, Yardley, PA (US); Raymond Kwong, Westhampton, NJ (US)

Correspondence Address: KENYON & KENYON LLP, 1500 K STREET N.W., SUITE 700, WASHINGTON, DC, 20005, US

(73) Assignee: Universal Display Corporation, Ewing, NJ (US)

(21) Appl. No.: 11/956,064

(22) Filed: Dec. 13, 2007

Related U.S. Application Data
(60) Provisional application No. 60/874,612, filed on Dec. 13, 2006.

(51) Int. Cl. C23C 16/18; C23C 16/448; A61L 9/02; H01L 27/32

(52) U.S. Cl. 427/96.8; 239/60; 427/252; 438/99

(57) ABSTRACT

Methods for evaporating (e.g., subliming) a material in the solid state. In one aspect, a mass of the material in the solid state is mixed with a plurality of packing units, wherein each of the packing units comprises an inert material. The structure of each of the packing units, or the structure of an aggregate of the packing units, comprises a plurality of non-smooth features. In one example, Pro-Pak™ metal meshes can be used, which have a plurality of various types of non-smooth features, including sharp edges, corners, and/or protrusions. These non-smooth features are believed to physically disrupt crust formation during the evaporation process. Also disclosed are other methods for evaporating a material and methods for fabricating an organic thin-film device.

### CROSS-REFERENCES

This application claims priority to U.S. Provisional Application Ser. No. 60/874,612 (filed 13 Dec. 2006), which is incorporated by reference herein in its entirety.

### TECHNICAL FIELD

The present invention relates to evaporation processes for solid phase materials.

### RESEARCH AGREEMENTS

The claimed inventions were made by, on behalf of, and/or in connection with one or more of the following parties to a joint university corporation research agreement: Princeton University, The University of Southern California, and the Universal Display Corporation. The agreement was in effect on and before the date the claimed inventions were made, and the claimed inventions were made as a result of activities undertaken within the scope of the agreement.

### BACKGROUND

There is currently considerable interest in the development of organic light-emitting devices (OLEDs) as a new generation of display technology, capable of providing high efficiencies, vivid colors, fast switching times, excellent viewing angles and ultra-thin form factors. However, in order to realize its commercial potential, improved manufacturing methods are required, including improved processes for vacuum sublimation of the metal complexes that are used as phosphorescent emitters. A key challenge in such processes is to achieve high yields while minimizing decomposition of the materials to be sublimed, especially in the case of the relatively high molecular weight metal complexes useful for phosphorescent OLED devices. We have observed that at long sublimation times (on the order of tens of hours) initially free-flowing powders of many such complexes tend to form a crust which serves as a barrier to sublimation. In order to maintain a constant sublimation rate in the presence of such a crust, it is necessarily to continually raise the sublimation temperature, which in turn tends to promote further decomposition, ultimately reducing the yield of sublimed material and potentially compromising the performance of the OLED device through the deposition of impurities. It is therefore one of the important objectives of OLED research to develop improved sublimation methods which minimize crusting during the sublimation of various types of materials including phosphorescent metal complexes.

### SUMMARY

In one aspect, the present invention provides a method for evaporating a material, comprising: (a) mixing a mass of the material in the solid state with a plurality of packing units, wherein each of the packing units comprises an inert material, and wherein the structure of each of the packing units or an aggregate of the packing units comprises a plurality of non-smooth features; and (b) evaporating at least a portion of the mass of the material.

In another aspect, the present invention provides a method of evaporating a material, comprising: (a) evaporating at least a portion of a mass of the material in the solid state at an environmental pressure of 10 torr or less; and (b) mechanically agitating the mass of the material during at least a portion of the evaporation.

In yet another aspect, the present invention provides a method of fabricating an organic thin-film device, comprising: (a) depositing a mass of a material onto a plurality of packing units, wherein each of the packing units comprises an inert material, and wherein the structure of each of the packing units or an aggregate of the packing units comprises a plurality of non-smooth features; (b) evaporating at least a portion of the mass of the material, in the solid state, that is on the packing units; (c) providing an electrode disposed over a substrate; and (d) forming a layer on a surface that is on or over the electrode by depositing the evaporated material onto the surface.

### BRIEF DESCRIPTION OF THE DRAWINGS

FIGS. 1A and 1B show individual Pro-Pak units (0.16 in², Monel).

FIG. 2 shows a demonstration of how void fraction is calculated.

FIG. 3A shows a frame used for holding packing units to form a structured packing assembly. FIG. 3B shows the frame of FIG. 3A with packing units attached.

FIG. 4 shows a structured packing assembly having an intersecting corrugated sheet structure.

FIG. 5A shows a perspective, see-through view of a mechanical agitation device that can be used in the present invention. FIG. 5B shows a cross-section view of the device of FIG. 5A.

FIG. 6 shows a perspective, see-through view of another mechanical agitation device that can be used in the present invention.

FIG. 7 shows a sublimation crucible containing a mass of compound 1a mixed with a plurality of packing units.

FIG. 8 shows the crucible and crystal monitor geometry inside the high vacuum chamber used in Example 1.

FIG. 9 shows a graph of the temperature rise observed during sublimation of compound 1a as a function of the rate of consumption of compound 1a.

### DETAILED DESCRIPTION

The present invention provides various methods involving the evaporation of a material in the solid state. The material being evaporated is any of the various materials which suffer from the problem of crusting during an evaporation process (e.g., sublimation). In some cases, the material comprises an organometallic complex; and in some cases, the material comprises a phosphorescent metal complex. As used herein, the term “evaporation” refers to any process by which a material (whether in solid or liquid phase) is converted to a gaseous state without undergoing any chemical reactions that result in a chemical change in the material. One example of evaporation is sublimation.

As such, in one aspect, the present invention provides a process for the evaporation of a material by mixing a mass of the material in the solid state with a plurality of packing units, wherein each of the packing units comprises an inert material. As used herein, the terms “inert material,” when referring to the packing units, means a material that is non-reactive with the material being evaporated.

The structure of each of the packing units, or the structure of an aggregate of the packing units, comprises a plurality of non-smooth features. The mass of the material may have a volume that is comparable to the total volume of the plurality of packing units. The use of such packing units having non-smooth features (e.g., having a plurality of sharp corners and edges) has been found to dramatically reduce the amount of crusting and reduce the temperature rate increase required to maintain a constant sublimation rate (on the order of ⅕th to 1/10th the rate of increase observed for control samples).

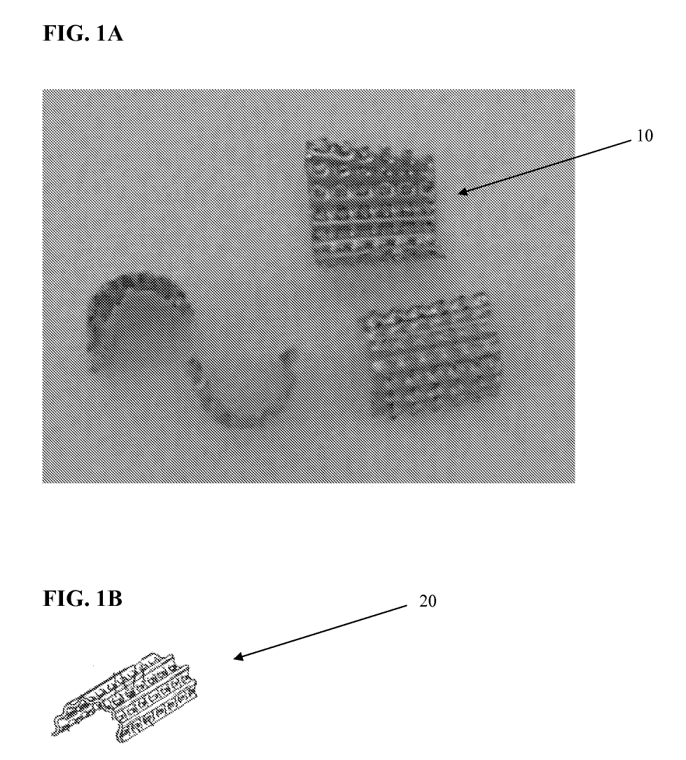

For example, when a sublimation crucible is loaded with strips of metal mesh (Pro-Pak™, 0.16 in², Monel) and sufficient compound 1a is added to almost cover the packing strips, significantly less discoloration was observed after 61 hrs, and the rate of increase of the sublimation temperature at comparable rates of sublimation was much less. As seen in FIGS. 1A and 1B, and as known to persons of ordinary skill in the art, Pro-Pak™ metal meshes 10 and 20 have a plurality of various types of non-smooth features, including sharp edges, corners, and protrusions.

Without wishing to be bound by theory, the inventors believe that these non-smooth features, which can have irregular shapes, act to physically disrupt crust formation by: (i) promoting loose packing and voids, which not only increase the surface area, but also gives a very mechanically weak 3-dimensional structure that tends to collapse and expose fresh surface as material is removed by sublimation; or (ii) jutting through any nascent crust to provide pathways by which vapors from underlying material to escape and to prevent the growing crust from forming a smooth interface that can maintain its integrity as the underlying material is sublimed as the crust settles; or (iii) a combination of these effects.

Where the material being evaporated is provided in powder form, the type of packing units can be selected according to the particle size of the powder. For example, one type of packing unit may be suited for a material in a very fine powder form (about 10 microns), and another type of packing unit may be suited for materials having large granules (about 500 microns).

In some cases, the volume ratios can be adjusted to provide the desired results. In this context, “volume” is intended to mean the simple volume, including free space, occupied by the packing units, the mass of the material being evaporated, or the mixture. In one embodiment, the void fraction of the mixture of packing units and the material being evaporated is at least 10%; and in some cases, at least 20%; and in some cases, at least 30%; and in some cases, at least 40%; and in some cases, at least 50%. By “void fraction”, we mean the percentage V_{f}=[(Vₘᵢₓₜᵤᵣₑ−Vₘₐₜₑᵣᵢₐₗ)/Vₘᵢₓₜᵤᵣₑ]×100, where Vₘᵢₓₜᵤᵣₑ is the volume of the mixture and Vₘₐₜₑᵣᵢₐₗ is the volume of the material being evaporated.

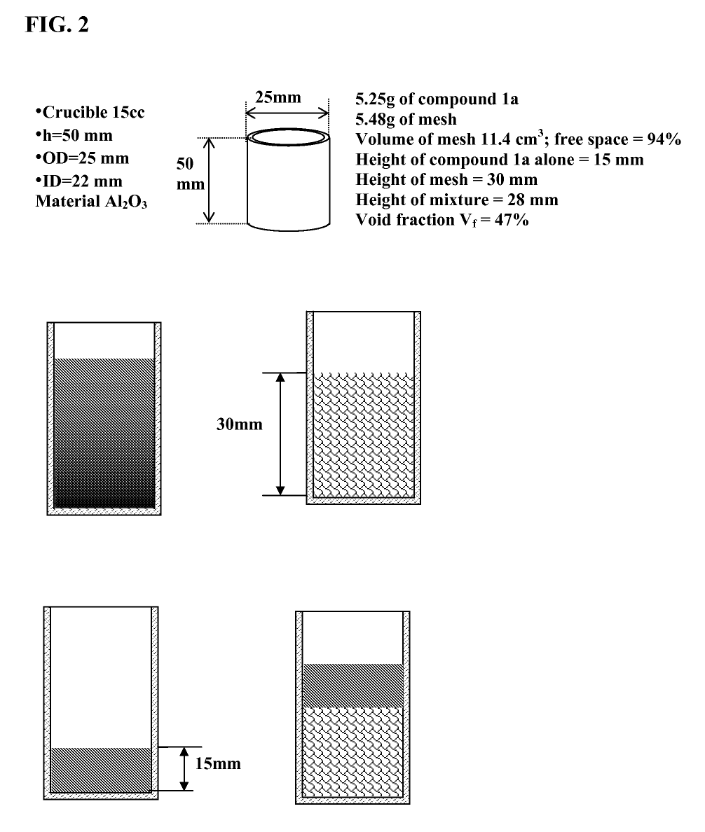

FIG. 2 illustrates how the void fraction is calculated. The simple volume of the metal mesh is 11.4 cm³, with 94% of this volume constituting free space. When the volume of the mesh, the volume of the compound, and the volume of the mixture (as calculated proportionate to their heights in a cylindrical container) are considered, the void fraction is determined to be 47%.

In certain embodiments, the packing units are selected from the group consisting of Penn State Packing or Pro-Pak™ (obtained from Aldrich). In certain embodiments, the inert material used in the packing units is a metal selected from the group consisting of the non-radioactive metals with atomic numbers greater than 21; and in some cases, greater than 40. Such metals can include: Cr, Mn, Fe, Co, or Ni. Such metals can also include: Re, Ru, Os, Rh, Ir, Pd, Pt, Cu, or Au. In some cases, the metal is Os, Ir, or Pt; and in some cases, the metal is Ir. In certain embodiments, the packing units may comprise thermally and chemically stable solid materials such as stainless steel (e.g., Pro-Pak™ Z210536 PAK), tantalum, molybdenum, tungsten, Hastelloy™, borosilicate glass (e.g. Pyrex™), boron nitride, aluminum oxide, or graphite. In certain embodiments, the inert material used in the packing units may comprise a material having a thermal conductivity of greater than 1 W/m·K; and in some cases, greater than 30 W/m·K; and in some cases, greater than 50 W/m·K.

In certain embodiments, the packing units are assembled into a structured packing assembly that acts to physically disrupt crust formation, either by (i) promoting loose packing and voids, which not only increase the surface area, but also gives a very mechanically weak 3-dimensional structure that tends to collapse and expose fresh surface as material is removed by sublimation; or (ii) jutting through any nascent crust to provide pathways by which vapors from underlying material to escape and to prevent the growing crust from forming a smooth interface that can maintain its integrity as the underlying material is sublimed as the crust settles; or (iii) a combination of these effects.

The structured packing assemblies of the present invention can be analogous to those used in distillation. For example, structured packing assemblies can include structured column packings formed using vertical sheets of corrugated thin gauge metal or metal mesh with the angle of the corrugations reversed in adjacent sheets to form an open honeycomb structure with inclined flow channels and a relatively high surface area. Additional perforations and surface texturing, including waffled, grooved, and smooth surfaces, can be used to facilitate vapor escape while providing a large surface area and potential to form high void content columns of powder upon initially charging the material to be evaporated to the structured packing. Such voids can help to promote the formation of mechanically weak powder masses that will collapse to expose new surface material during evaporation. Several slabs of packing units can be stacked on top of each other, with the columns offset or the column axes rotated from one slab to the next to facilitate the desired high void content loading and make it impossible for the material to be evaporated to densely pack upon collapse. Examples of companies that provide such packing units and assemblies for distillation purposes include: AceChemPack, Koch-Glitsch, Sulzer, and ACS Industries, Inc.

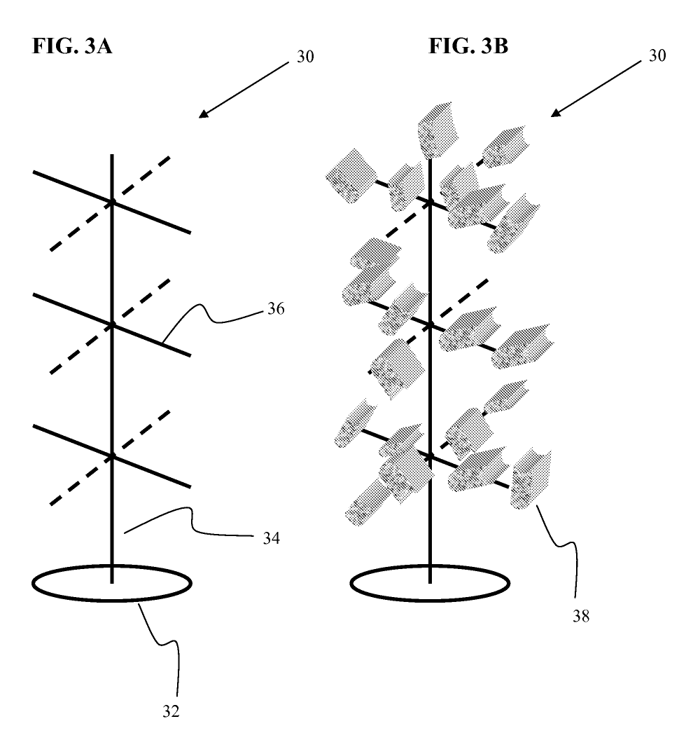

In another example, as shown in FIGS. 3A and 3B, the structured packing assembly can have a Christmas tree-shaped framework 30, which is designed to promote loose packing, void formation, and disrupt crust formation, while at the same time allowing for a reproducible set up of the deposition source from experiment (or manufacturing run) to experiment (or manufacturing run). Referring to FIG. 3A, the Christmas tree structure has a base 32, a trunk 34 extending vertically from base 32, and a number of branches 36 extending from trunk 34. FIG. 3B shows individual packing units 38 (Pro-Pak™, 0.16 in², Monel) that are attached to framework 30. Many other shapes and sizes (e.g., total size, surface area, or void space size) could be used for the packing units and/or structured packing assembly (e.g., honeycombs, corkscrew shapes, etc.)

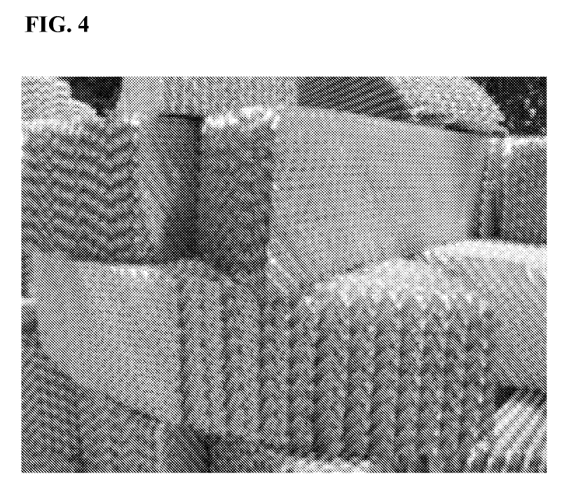

Other frame shapes and packing arrangements are possible. Connecting the individual packing units together, depending on the composition of the pieces (e.g., metals and alloys), may also promote increased thermal conductivity within the source, which may be beneficial in controlling the deposition rate of the material being evaporated as a function of time. The individual packaging units could also be fused together into one assembly that would fit into a crucible. In another example, FIG. 4 shows a structured packing assembly comprising intersecting elements of corrugated metal sheet.

The various types of evaporation processes used in the fabrication of OLEDs and other organic thin film devices can take place under different environmental pressures. For example, organic vapor phase deposition (OVPD) generally takes place at an environmental pressure of around 1 torr. Vacuum thermal evaporation (VTE) generally takes place at an environmental pressure of 10⁻⁶-10⁻⁸ torr. As such, in certain embodiments, the evaporation takes place at an environmental pressure of less than 10 torr; and in some cases, less than 1 torr; and in some cases, less than 10⁻² torr; and in some cases, less than 10⁻⁵ torr.

In certain embodiments, the evaporated material can be used in the fabrication of an organic thin-film device. For example, the fabrication may involve the formation of a layer by depositing the evaporated material onto a surface. The organic thin-film device may be an OLED; and the layer may be an electroluminescent layer, a charge-transporting layer (e.g., hole-transporting or electron-transporting), or a blocking layer.

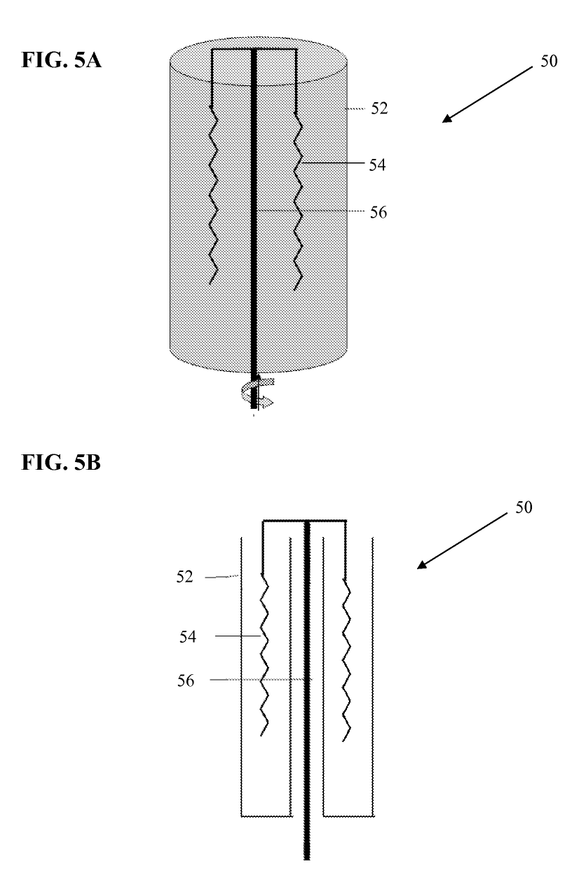

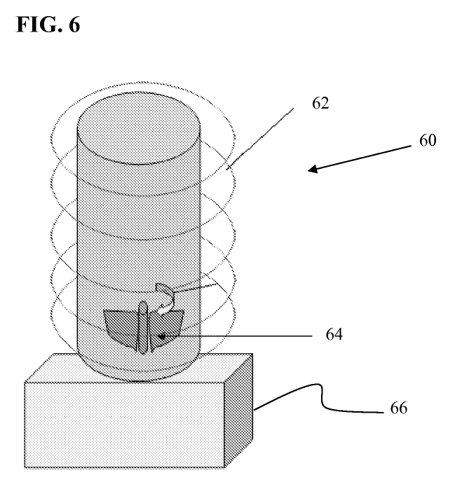

In another aspect, the present invention provides a process for the evaporation of a material by mechanically agitating the material, intermittently or continuously, while the material is being evaporated. For examples, FIGS. 5A and 5B show a device 50 for mechanical agitation comprising a rotating axle 56 connected to a stirrer 54 and a heating element 52. Rotation of rotating axle 56 causes the rotation of stirrer 54, which agitates the material being evaporated. In another example, FIG. 6 shows another device 60 for mechanical agitation comprising a stirrer bar 64, a magnetic stirrer base 66, and a coil heating element 62. A rotating magnetic field created by magnetic stirrer base 66 causes stirrer bar 64 to spin, which agitates the material being evaporated.

In certain embodiments, the material being evaporated may further be mixed with the packing units or structured packing assemblies described above. In certain embodiments, the evaporation takes place at the environmental pressures described above.

In another aspect, the present invention provides a method for the fabrication of an organic thin-film device by depositing a mass of the material to evaporated onto a plurality of the packing units as described above. This material that is deposited onto the packing units then serves as the source material for fabricating the organic thin-film device by evaporation or sublimation techniques (e.g., by evaporating the material off of the packing units and depositing onto a surface in the process of fabricating the organic thin-film device). The mass of the material may be deposited onto the packing units in various ways, including the use of another evaporation or sublimation process, or by crystallizing the material onto the packing units. Further, the above-described packing units and process parameters may be used in this method for fabricating an organic thin-film device.

### EXAMPLES

Specific representative embodiments of the invention will now be described, including how such embodiments may be made. It is understood that the specific methods, materials, conditions, process parameters, apparatus and the like do not necessarily limit the scope of the invention.

### Comparative Example 1

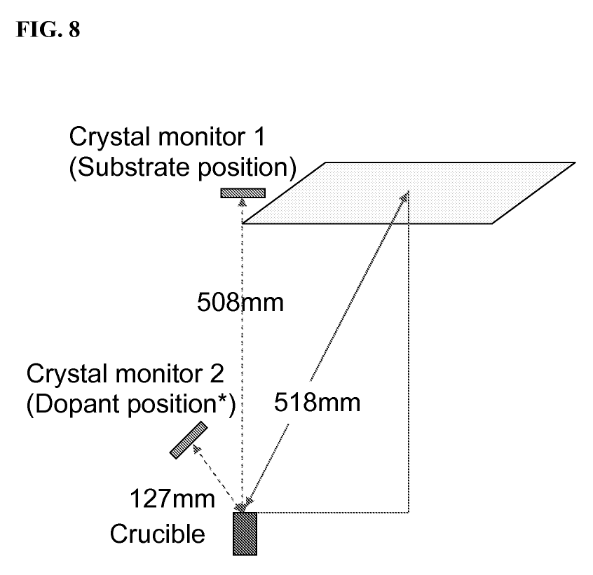

An alumina crucible (Luxel Alumina RADAK™ II P/N 20300-1) was filled with 10.06 g of compound 1a. The crucible was loaded into a high vacuum chamber and the chamber was pumped down to a vacuum level <10⁻⁶ torr. The crucible was then heated by a coil heater (Luxel RADAK™ II) surrounding the sides of the crucible. Sublimation of compound 1a from the crucible was monitored by a thickness monitor. A constant sublimation rate was maintained by adjusting the power through the heater coil. The rate was set at 0.07 Å/s for this test (the calculated rate at substrate position) as monitored by a crystal monitor in position 2 (see FIG. 8). Compound 1a was sublimed for 130 hours before the experiment was stopped. During the first 64 hours, the rate of increase of the temperature required to maintain a constant sublimation rate was 0.19° C./h. The consumption rate from the crucible was 0.016 g/h during this 130 hour experiment. After 64 hours, the experiment was interrupted, and upon inspection, discoloration was observed at the surface of compound 1a, indicating possible material decomposition during the extended sublimation time.

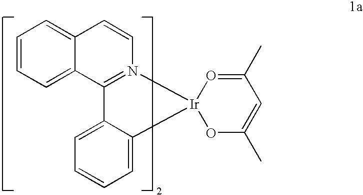

### Example 1

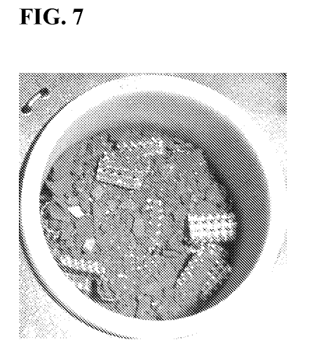

5.39 g of metal mesh packing strips (Pro-Pak™, 0.16 in², Monel) were loaded into a sublimation crucible (Luxel Alumina RADAK™ II P/N 20300-1). 5.70 g of compound 1a was added to cover the metal mesh packing. As shown in FIG. 7, the crucible was agitated to mix compound 1a with the metal mesh packing. The crucible was loaded into a high vacuum chamber and the chamber was pumped down to a vacuum level <10⁻⁶ torr. The crucible was then heated by a coil heater (Luxel RADAK™ II) surrounding the sides of the crucible. Sublimation of compound 1a from the crucible was monitored by a thickness monitor. A constant sublimation rate was maintained by adjusting the power through the heater coil. The rate was set at 0.08 Å/s for this test (the calculated rate at the substrate position) as monitored by a crystal monitor in position 2 (see FIG. 8). Compound 1a was sublimed for 61 hours continuously before the experiment was stopped. During the first 34 hours, no temperature increase of the crucible was required to maintain a constant deposition rate. After 34 hours, the rate of increase of the temperature required to maintain a constant sublimation rate was 0.05° C./h (the average rate increase of the temperature required to maintain a constant sublimation rate over 61 hours was 0.02° C./h). The consumption rate from the crucible was 0.017 g/h during this experiment. After 61 hours, heating of the crucible was stopped. Thus, in comparison to Comparative Example 1, a slower rate of temperature increase was needed to maintain a comparable sublimation rate. Further, upon inspection of the crucible, there was significantly less discoloration at the surface in comparison to the result obtained in Comparative Example 1.

### Comparative Example 2

An alumina crucible (Luxel Alumina RADAK™ II P/N 20300-1) was filled with 1.00 g of compound 1a. The crucible was loaded into a high vacuum chamber and the chamber was pumped down to a vacuum level <10⁻⁶ torr. The crucible was then heated by a coil heater (Luxel RADAK™ II) surrounding the sides of the crucible. Sublimation of compound 1a from the crucible was monitored by a thickness monitor. A constant sublimation rate was maintained by adjusting the power through the heater coil. The rate was set at 0.25 Å/s (the calculated rate at substrate position) for this test, as monitored by a crystal monitor in position 2 (see FIG. 8). Compound 1a was sublimed for 4.8 hours before the experiment was stopped. During the experiment, the rate of increase of the temperature required to maintain a constant sublimation rate was 1.04° C./h. The consumption rate from the crucible was 0.081 g/h during this 4.8 hour experiment. After 4.8 hr, the experiment was stopped. Upon inspection of the crucible, the surface had become discolored, similar to the result obtained for Comparative Example 1.

### Example 2

6.35 g of metal mesh packing strips (Pro-Pak™, 0.16 in², Monel) were loaded into an empty sublimation crucible (Luxel Alumina RADAK™ II P/N 20300-1). 5.98 g of compound 1a was added to cover the packing. The crucible was agitated to mix compound 1a with the metal mesh packing. The crucible was loaded into a high vacuum chamber and the chamber was pumped down to a vacuum level <10⁻⁶ torr. The crucible was then heated by a coil heater (Luxel RADAK II™) surrounding the sides of the crucible. Sublimation of compound 1a from the crucible was monitored by a thickness monitor. A constant sublimation rate was maintained by adjusting the power through the heater coil. The rate was set at 0.2 Å/s for this test (the calculated rate at substrate position), as monitored by a crystal monitor in position 1 (see FIG. 8). Compound 1a was sublimed for 64 hours continuously before the experiment was stopped. The rate of increase of the temperature required to maintain a constant sublimation rate was 0.19° C./h over the 64 hour run. The consumption rate from the crucible was 0.07 g/h during this experiment. After 64 hours, heating of the crucible was stopped. Upon inspection of the crucible after removal from the vacuum chamber, there was significantly less discoloration at the surface in comparison to the result obtained in Comparative Example 2.

### Comparative Example 3

An alumina crucible (Luxel Alumina RADAK™ II P/N 20300-1) was filled with 1.00 g of compound 1a. The crucible was loaded into a high vacuum chamber and the chamber was pumped down to a vacuum level <10⁻⁶ torr. The crucible was then heated by a coil heater (Luxel RADAK™ II) surrounding the sides of the crucible. Sublimation of compound 1a from the crucible was monitored by a thickness monitor. A constant sublimation rate was maintained by adjusting the power through the heater coil. The rate was set at 0.50 Å/s (the calculated rate at substrate position) for this test, as monitored by a crystal monitor in position 2 (see FIG. 8). Compound 1a was sublimed for 5.3 hours before the experiment was stopped. During the experiment, the rate of increase of the temperature required to maintain a constant sublimation rate was 1.92° C./h. The consumption rate from the crucible was 0.110 g/h during this 5.3 hour experiment. After 5.3 hr, the experiment was stopped.

### Example 3

6.11 g of metal mesh packing strips (Pro-Pak™, 0.16 in², Monel) were loaded into an empty sublimation crucible (Luxel Alumina RADAK™ II P/N 20300-1). 5.57 g of compound 1a was added to cover the packing. The crucible was agitated to mix compound 1a with the metal mesh packing. The crucible was loaded into a high vacuum chamber and the chamber was pumped down to a vacuum level <10⁻⁶ torr. The crucible was then heated by a coil heater (Luxel RADAK™ II) surrounding the sides of the crucible. Sublimation of compound 1a from the crucible was monitored by a thickness monitor. A constant sublimation rate was maintained by adjusting the power through the heater coil. The rate was set at 0.53 Å/s for this test (the calculated rate at substrate position), as monitored by a crystal monitor in position 1 (see FIG. 8). Compound 1a was sublimed for 17 hours continuously before the experiment was stopped. The rate of increase of the temperature required to maintain a constant sublimation rate was 0.65° C./h over the 17 hour run. The consumption rate from the crucible was 0.175 g/h during this experiment. After 16 hours, heating of the crucible was stopped. Upon inspection of the crucible, there was significantly less discoloration at the surface in comparison to the result obtained in Comparative Example 3.

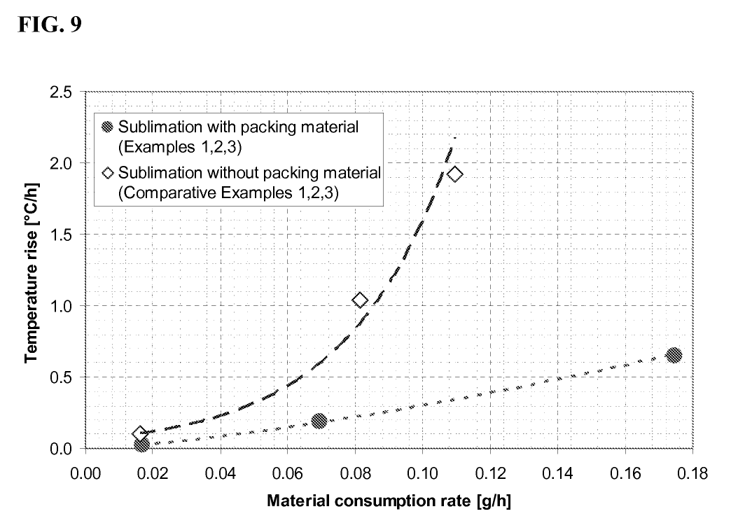

FIG. 9 shows a graph of the temperature rise observed during sublimation of compound 1a as a function of the rate of consumption of compound 1a in the above examples. Table 1 below summarizes the sublimation data obtained from the above examples. This data demonstrates that methods of the present invention can allow for a slower rate of temperature increase to maintain a comparable sublimation rate.

TABLE 1

| Evaporation | Example | Example | Example | Comparative | Comparative | Comparative |
|---|---|---|---|---|---|---|
| source | 1 | 2 | 3 | Example 1 | Example 2 | Example 3 |
| Evaporation | 0.08 | 0.20 | 0.53 | 0.07 | 0.25 | 0.50 |
| rate (Å/s) | | | | | | |
| Run time (hr) | 61 | 64 | 17 | 64 (130) | 4.8 | 5.3 |
| Mesh amount (g) | 5.39 | 6.35 | 6.11 | — | — | — |
| Compound 1a | 5.70 | 5.98 | 5.57 | 10.06 | 1.00 | 1.00 |
| loaded (g) | | | | | | |
| Compound 1a | 1.03 | 4.45 | 2.97 | 2.11 | 0.39 | 0.57 |
| consumed (g) | | | | | | |
| Starting | 212.5 | 251 | 267 | 238 | 243 | 249 |
| evaporation | | | | | | |
| temperature (° C.) | | | | | | |
| Final evaporation | 214 | 263 | 278 | 250 | 248 | 259 |
| temperature (° C.) | | | | | | |
| Material | 0.017 | 0.070 | 0.175 | (0.016*) | 0.081 | 0.110 |
| consumption | | | | | | |
| (g/h) | | | | | | |
| Evaporation | 0.02 | 0.19 | 0.65 | 0.19 | 1.04 | 1.92 |
| temperature rise | | | | | | |
| rise (° C./h) | | | | | | |

*consumption rate for 130 hr

The foregoing description and examples have been set forth merely to illustrate the invention and are not intended to be limiting. Each of the disclosed aspects and embodiments of the present invention may be considered individually or in combination with other aspects, embodiments, and variations of the invention. Modifications of the disclosed embodiments incorporating the spirit and substance of the invention may occur to persons skilled in the art and such modifications are within the scope of the present invention.

What is claimed is:

1. A method for evaporating a material, comprising
mixing a mass of the material in the solid state with a plurality of packing units, wherein each of the packing units comprises an inert material, and wherein the structure of each of the packing units or an aggregate of the packing units comprises a plurality of non-smooth features; and
evaporating at least a portion of the mass of the material.

2. The method of claim 1, wherein the non-smooth features comprise edges, corners, or projections.

3. The method of claim 2, wherein the non-smooth features are sharp.

4. The method of claim 1, wherein the non-smooth features comprise a mesh structure.

5. The method of claim 1, wherein the non-smooth features comprise a plurality of perforations.

6. The method of claim 1, wherein the non-smooth features comprise corrugations or grooves.

7. The method of claim 1, wherein the inert material in the packing units has a thermal conductivity of 1 W/m·K or greater.

8. The method of claim 7, wherein the inert material in the packing units has a thermal conductivity of 30 W/m·K or greater.

9. The method of claim 8, wherein the inert material in the packing units has a thermal conductivity of 50 W/m·K or greater.

10. The method of claim 1, wherein the step of evaporating comprises subliming the material.

11. The method of claim 1, wherein the step of evaporating comprises heating the mass of the material.

12. The method of claim 1, wherein the step of evaporating takes place at an environmental pressure of 10 torr or less.

13. The method of claim 12, wherein the environmental pressure is 1 torr or less.

14. The method of claim 13, wherein the environmental pressure is 10⁻² torr or less.

15. The method of claim 14, wherein the environmental pressure is 10⁻⁵ torr or less.

16. The method of claim 1, wherein the inert material in the packing units comprises a non-radioactive metal having an atomic number greater than 21.

17. The method of claim 16, wherein the non-radioactive metal has an atomic number greater than 40.

18. The method of claim 17, wherein the non-radioactive metal is selected from the group consisting of: Re, Ru, Os, Rh, Ir, Pd, Pt, Cu, and Au.

19. The method of claim 17, wherein the inert material in the packing units is an alloy.

20. The method of claim 1, wherein the material being evaporated comprises an organometallic complex.

21. The method of claim 20, wherein the organometallic complex is a phosphorescent organometallic complex.

22. The method of claim 1, wherein the void fraction of the mixture of the packing units and the mass of the organometallic complex is at least 10%.

23. The method of claim 22, wherein the void fraction of the mixture of the packing units and the mass of the organometallic complex is at least 20%.

24. The method of claim 23, wherein the void fraction of the mixture of the packing units and the mass of the organometallic complex is at least 30%.

25. The method of claim 24, wherein the void fraction of the mixture of the packing units and the mass of the organometallic complex is at least 40%.

26. The method of claim 25, wherein the void fraction of the mixture of the packing units and the mass of the organometallic complex is at least 50%.

27. The method of claim 1, wherein the packing units are arranged randomly in the mixture.

28. The method of claim 1, wherein the packing units are assembled into a structured packing assembly.

29. The method of claim 28, wherein the structured packing assembly includes a frame, and wherein the packing units are connected to the frame.

30. The method of claim 29, wherein the frame has a vertical axis, and wherein the packing units at one vertical level of the frame are arranged in an offset position in relation to the packing units at an adjacent vertical level of the frame.

31. The method of claim 28, wherein at least one of the packing units is fused to another packing unit.

32. The method of claim 28, wherein the structured packing assembly forms corrugated sheets.

33. The method of claim 1, further comprising mechanically agitating the mixture of the packing units and the mass of the material during at least a portion of the evaporation.

34. The method of claim 1, further comprising using the evaporated material in the fabrication of an organic thin-film device by:
providing an electrode disposed over a substrate; and
forming a layer on a surface that is on or over the electrode by depositing the evaporated material onto the surface.

35. The method of claim 32, wherein the organic thin-film device is an organic light-emitting device, and wherein the layer is an electroluminescent layer.

36. The method of claim 32, wherein the organic thin-film device is an organic light-emitting device, and wherein the layer is a charge-transporting layer or blocking layer.

37. A method for evaporating a material, comprising:
evaporating at least a portion of a mass of the material in the solid state at an environmental pressure of 10 torr or less; and
mechanically agitating the mass of the material during at least a portion of the evaporation.

38. The method of claim 37, wherein the step of evaporating comprises heating the mass of the material

39. The method of claim 37, wherein the step of mechanically agitating comprises stirring the mass of the material.

40. The method of claim 37, wherein the mechanical agitation is performed intermittently.

41. The method of claim 37, wherein the mechanical agitation is performed continuously.

42. The method of claim 37, further comprising, prior to the step of evaporating, mixing the mass of the material with a plurality of packing units, wherein each of the packing units comprises an inert material, and wherein the structure of each of the packing units or an aggregate of the packing units comprises a plurality of non-smooth features.

43. The method of claim 42, wherein the step of mechanically agitating includes agitating the packing units.

44. The method of claim 37, wherein the material comprises an organometallic complex.

45. The method of claim 44, wherein the organometallic complex is a phosphorescent organometallic complex.

46. The method of claim 37, wherein the environmental pressure is 1 torr or less.

47. The method of claim 46, wherein the environmental pressure is 10⁻² torr or less.

48. The method of claim 47, wherein the environmental pressure is 10⁻⁵ torr or less.

49. A method for fabricating an organic thin-film device, comprising:
depositing a mass of a material onto a plurality of packing units, wherein each of the packing units comprises an inert material, and wherein the structure of each of the packing units or an aggregate of the packing units comprises a plurality of non-smooth features;
evaporating at least a portion of the mass of the material, in the solid state, that is on the packing units;
providing an electrode disposed over a substrate; and
forming a layer on a surface that is on or over the electrode by depositing the evaporated material onto the surface.

50. The method of claim 49, wherein the organic thin-film device is an organic light-emitting device, and wherein the layer is an electroluminescent layer.

51. The method of claim 49, wherein the organic thin-film device is an organic light-emitting device, and wherein the layer is a charge-transporting layer or blocking layer.

52. The method of claim 49, wherein the step of depositing the mass of the material is performed by crystallizing the material onto the packing units.

53. The method of claim 49, wherein the step of depositing the mass of the material is performed by evaporating the material and depositing the evaporated material onto the packing units.

54. The method of claim 49, wherein the material comprises an organometallic complex.
